(19) United States
(12) Patent Application Publication
(10) Pub. No.: US 2019/0142721 A1
(43) Pub. Date: May 16, 2019

(54) COMPOSITION CONTAINING SUBSTANCE FOR REGULATING EXPRESSON OF ABH ANTIGENS

(71) Applicant: AMOREPACIFIC CORPORATION, Seoul (KR)

(72) Inventors: Yong Deog HONG, Yongin-si (KR); Kyum Son KIM, Yongin-si (KR); Jun Seong PARK, Yongin-si (KR); Sang Hoon HAN, Yongin-si (KR)

(73) Assignee: AMOREPACIFIC CORPORATION, Seoul (KR)

(21) Appl. No.: 16/241,414

(22) Filed: Jan. 7, 2019

Related U.S. Application Data
(62) Division of application No. 15/519,334, filed on Apr. 14, 2017.

(30) Foreign Application Priority Data
Oct. 15, 2014 (KR) 10-2014-0138912
Oct. 15, 2014 (KR) 10-2014-0138913

(51) Int. Cl. A61K 8/49; A61Q 19/00; G01N 33/50; G01N 33/80; A61Q 19/08; A61K 8/37

(52) U.S. Cl. CPC A61K 8/498; A61Q 19/008; G01N 33/5023; G01N 33/80; G01N 2800/20; A61Q 19/08; A61K 8/375; A61Q 19/00; A61K 2800/74; G01N 33/5044

(57) ABSTRACT

A composition containing a material for regulating the expression of ABH antigens and, more specifically, to a composition capable of: controlling sebum production and alleviating skin trouble by regulating the expression of ABH antigens; preventing skin pore enlargement by providing antioxidant effects; and defending against skin irritation production and method of using the composition are disclosed.

### CROSS REFERENCE TO RELATED APPLICATIONS

This application is a Divisional Application of U.S. application Ser. No. 15/519,334, filed Apr. 14, 2017, which is a National Stage of International Application No. PCT/KR2014/009742 filed Oct. 16, 2014, claiming priority based on Korean Patent Application No. 10-2014-0138912 filed Oct. 15, 2014 and Korean Patent Application No. 10-2014-0138913 filed Oct. 15, 2014, the contents of all of which are incorporated herein by reference in their entirety.

### TECHNICAL FIELD

The present invention relates to a composition for containing a substance that regulates the expression of ABH antigen. More specifically, the present invention relates to a composition capable of: controlling sebum production and improving skin troubles by regulating the expression of ABH antigen; preventing skin pore enlargement by providing antioxidant effects; and defending against skin irritation production.

### BACKGROUND OF ART

Blood group antigens refer to a structure having specific antigenicity expressed in glycoproteins or glycolipids on the surface of erythrocytes in the blood. Typically, there are ABO blood group antigens (ABH antigens), Lewis blood group antigens, and the like, and the blood group is determined according to the glycosylated terminal structure of the specific structure. ABO blood group antigens and Lewis blood antigens are not expressed only in red blood cells, but are expressed in various parts of the human body. Especially, ABO blood group antigens are known to be expressed even in the epithelium of the body such as esophagus, stomach, small intestine, and it is expressed in the granular layer of the epidermis in the skin.

The expression of such ABO blood group antigen in the granular layer of the epidermis appears in the outermost layer of the skin in an anatomical position, and thus is closely related to skin-related diseases, especially inflammatory diseases.

As such, ABO blood group antigens are very important antigens that are mainly responsible for rejection of transfusion and organ transplantation, but since their discovery in 1900, there has been little research on physiological functions other than rejection.

### DETAILED DESCRIPTION OF THE INVENTION

### Technical Problem

In this regard, the present inventors found that the regulation of ABH antigen expression is related to control of sebum, improvement of skin troubles, prevention of skin pore enlargement and the like, thereby completing the present invention.

Therefore, an object of the present invention is to provide a composition effective for controlling sebum or improving skin troubles by controlling the expression of ABH antigens.

Another object of the present invention is to provide a composition comprising a substance effective for reducing skin pores, preventing enlargement of skin pores and preventing skin aging by controlling the expression of ABH antigen.

### Technical Solution

In order to achieve these objects, the present invention provides a composition for regulating sebum comprising, as an active ingredient, a substance that regulates the expression of ABH antigen.

The present invention also provides a composition for improving skin troubles comprising, as an active ingredient, a substance that regulates the expression of ABH antigen.

The present invention also provides a composition for reducing skin pores comprising, as an active ingredient, a substance that regulates the expression of ABH antigen.

Further, the present invention also provides a composition for preventing skin pore enlargement comprising, as an active ingredient, a substance that regulates the expression of ABH antigen.

In addition, the present invention also provides a composition for preventing skin aging comprising, as an active ingredient, a substance that regulates the expression of ABH antigen.

### Advantageous Effects

The composition of the present invention provides the expression of ABH antigen to thereby provide excellent sebum control or skin trouble improving effects, and also reduces skin pores through active oxygen eliminating and collagen synthesis promotion, and further is very effective for defending against skin irritation production due to excellent antioxidative power.

### BRIEF DESCRIPTION OF DRAWINGS

FIG. 1 shows the structure of ABH antigen and Lewis blood group antigen.

FIG. 2 shows that the expression of B antigen from HaCaT cell line is increased by a substance that regulates the expression of ABH antigen.

### DETAILED DESCRIPTION OF THE EMBODIMENTS

The present invention relates to a composition comprising, as an active ingredient, a substance that regulates the expression of ABH antigen.

In particular, the composition of the present invention exhibits sebum control or skin trouble improving effects by controlling the expression of ABH antigen.

Further, the composition of the present invention exhibits the effects of reducing skin pores, preventing skin pore enlargement or preventing skin aging by controlling the expression of ABH antigen.

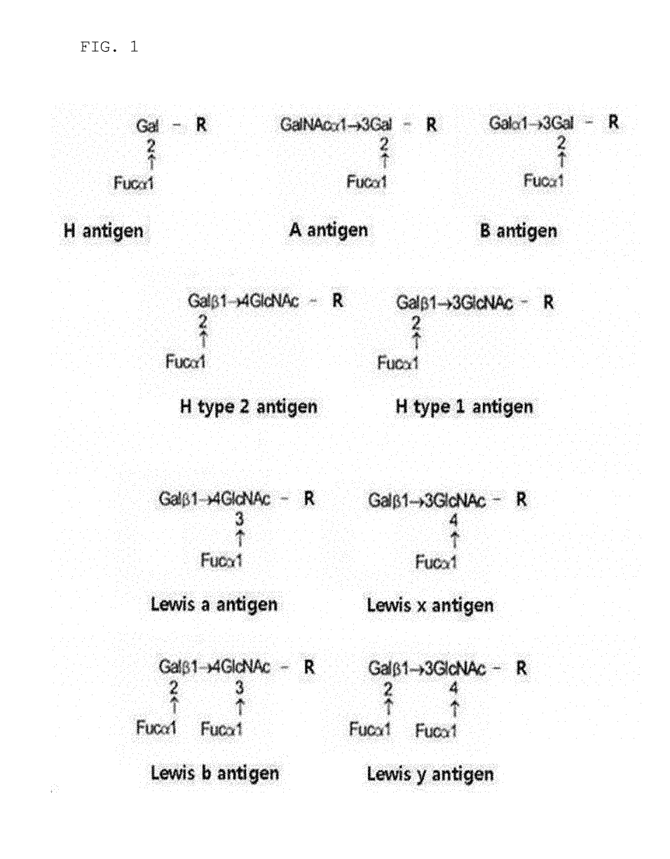

As used herein, the term “ABH antigen” refers to a structure having specific antigenicity expressed in glycoproteins or glycolipids on the surface of erythrocytes in the blood. Typically, the ABH antigen is used to have been including all aggregates of ABH antigen analogs such as ABH antigen and Lewis blood group antigen shown in FIG. 1. The ABH antigen analogue is a substance to which a monosaccharide, an amino acid, or the like is further bound, and means a substance having the same function as the original function of the ABH antigen. The structure of ABH antigen and Lewis blood group antigen is shown in FIG. 1.

As used herein, the term “active ingredient” refers to an ingredient that alone exhibits the desired activity or that can exhibit the activity in combination with a carrier having no activity by itself.

In the composition of the present invention, the substance for regulating the expression of the ABH antigen includes a substance that increases the expression of the ABH antigen. Specifically, the increase in the expression of ABH antigens is shown through an increase in the expression of B antigen in HaCaT cell line.

In the composition of the present invention, the substance for regulating the expression of the ABH antigen includes at least one compound selected from the group consisting of 1,3-dicaffeoylquinic acid (chemical formula 1), 1,5-dicaffeoylquinic acid (chemical formula 2) and amentoflavone (chemical formula 3), a derivative thereof, or a pharmaceutically acceptable salt thereof.

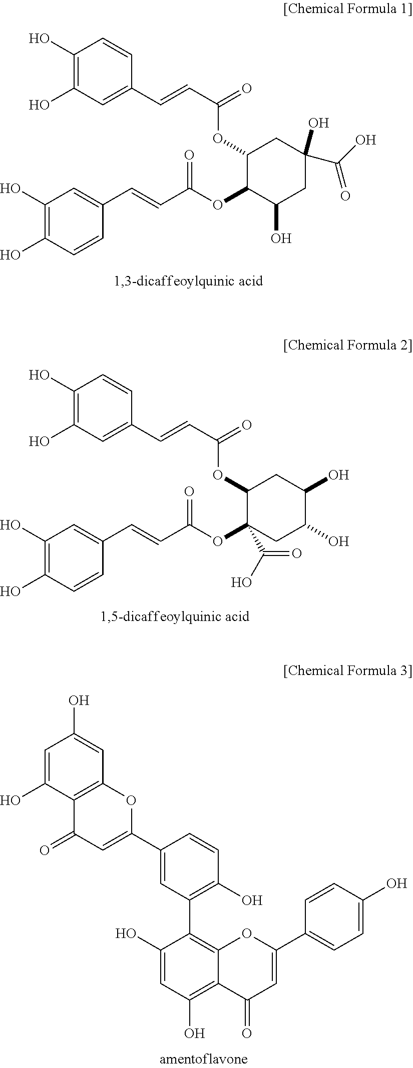

As used herein, the term “derivative” means all compounds that is changed to other substituent at a substitutable position of the above-mentioned compounds, and the type of such substituents is not limited.

As used herein, “pharmaceutically acceptable” means approved by a regulatory agency of the government or an international organization or listed in the Pharmacopoeia or other generally recognized pharmacopoeia for use in animals, more specifically in humans, since significant toxic effect can be avoided when used with a common medicinal dosage.

As used herein, “pharmaceutically acceptable salt” refers to a salt which is pharmaceutically acceptable and exhibits the desired pharmacological activity of its parent compound. The salt may include (1) an acid addition salt formed from an inorganic acid such as hydrochloric acid, hydrobromic acid, sulfuric acid, nitric acid, phosphoric acid, etc.; or formed from an organic acid such as acetic acid, propionic acid, hexanoic acid, cyclopentane propionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tert-butylacetic acid, lauryl sulfuric acid, gluconic acid, glutamic acid, hydroxynaphthoic acid, salicylic acid, stearic acid or muconic acid or; (2) a salt formed as an acidic proton present in the parent compound is replaced.

The composition of the present invention may contain a substance that regulates the expression of ABH antigen in an amount of 0.001% by weight to 20% by weight based on the total weight of the composition.

When the substance that regulates the expression of the ABH antigen is used within the above range, it is suitable for exhibiting the intended effect of the present invention and also can satisfy both stability and safety of the composition and further is useful in terms of cost effectiveness. From the above-mentioned viewpoint, the composition of the present invention may contain in an amount of 0.005 wt % to 19.5 wt %, 0.01 wt % to 19 wt %, 0.015 wt % to 18.5 wt %, 0.02 wt % to 18 wt %, 0.025 wt % to 17.5 wt %, 0.03 wt % to 17 wt %, 0.035 wt % to 16.5 wt %, 0.04 wt % to 16 wt % or 0.045 wt % to 15.5 wt % based on the total weight of the composition.

In the composition for controlling sebum according to an aspect of the present invention, the composition can inhibit the expression of 5α-reductase. Specifically, the composition of the present invention may interfere with the expression of 5α-reductase gene and so inhibit or suppress the expression, or inhibit the activity of 5α-reductase protein and interfere with its action.

In addition, in the composition for reducing skin pores or preventing enlargement of skin pore according to another aspect of the present invention, the composition can promote active oxygen elimination and collagen synthesis to reduce skin pores, prevent skin pore enlargement or skin aging, and further inhibit the production of active oxygen species and inhibit skin inflammation, thereby defending against the production of skin irritation.

In one aspect of the invention, the composition may be a cosmetic composition.

The formulation of the cosmetic composition is not particularly limited, but may be selected appropriately depending on desired purposes. For example, it may be formulated in one or more forms selected from the group consisting of a softening skin lotion (skin lotion or milk lotion), a skin nutrition lotion, an essence, a nutrition cream, a massage cream, a pack, a gel, an eye cream, an eye essence, a cleansing cream, a cleansing foam, a cleansing water, a powder, a body lotion, a body cream, a body oil and a body essence, but is not limited thereto.

In addition, the cosmetic composition may be used as an external preparation for skin in the form of ointment, patch, or the like.

The cosmetic composition according to the present invention may be provided in the form of any formulation suitable for topical application. For example, it may be provided in the form of solution, oil-in-water emulsion, water-in-oil emulsion, suspension, solid, gel, powder, paste, foam or aerosol. The composition for these formulations can be prepared according to the methods commonly employed in the art.

The cosmetic composition according to the present invention may include, in addition to the above substance, other ingredients providing synergic effect without negatively affecting the desired effect. In addition, the cosmetic composition according to the present invention may further include a moisturizing agent, an emollient agent, an ultraviolet absorber, an antiseptic, a sterilizer, an antioxidant, a pH adjuster, an organic and inorganic pigment, a fragrance, a cold sensing agent or an antiperspirant. The mixed amount of those ingredients may be easily determined by those skilled in the art within the ranges not deteriorating the purpose and effect of the present disclosure. The mixed amount may be 0.01 wt % to 5 wt %, specifically 0.01 wt % to 3 wt %, based on the total weight of the composition.

In the composition according to one aspect of the present invention, the composition can be a pharmaceutical composition.

The formulation of the pharmaceutical composition according to the present invention may be solution, suspension, emulsion, gel, drip, suppository, patch or spray, but is not limited thereto. These formulations may be prepared easily according to the methods commonly employed in the art and may include an excipient, a hydrating agent, an emulsification accelerator, a suspending agent, a salt or buffer for adjusting osmotic pressure, a coloring agent, a flavor, a stabilizer, an antiseptic, a preservative or other commonly used adjuvants, if desired.

The active ingredient of the pharmaceutical composition of the present invention may vary according to the patient's age, sex, weight, pathology state and severity, administration route, or prescriber's judgment. Suitable dosage may be determined by one of ordinary skill in the art based on the above-mentioned factors, and the daily dose may be, but is not limited to, 0.000025 mg/g/day to 0.025 mg/g/day, more specifically 0.00025 mg/g/day to 0.01 mg/g/days.

The pharmaceutical composition according to the present disclosure may be administered orally or transdermally, but is not limited thereto.

In addition, the present invention provides a screening method of a substance that regulates the expression of ABH antigen, comprising the steps of:
1) Confirming the expression level of the ABH antigen expressed in test skin cells;
2) treating the test skin cells with a candidate substance;
3) confirming the expression level of the ABH antigen from the cells of step 2); and
4) comparing the results of steps 1) and 3) above to determine whether it is a substance that increases the expression of the ABH antigen;

In the screening method according to one aspect of the present invention, the substance that regulates the expression of the ABH antigen is a substance that inhibits sebum production and alleviates skin troubles.

In addition, in the screening method according to another aspect of the present invention, the substance that regulates the expression of the ABH antigen is a substance having a skin pore reduction activity or a skin pore enlargement inhibiting activity or a skin aging-inhibitory activity.

In the method of the present invention, the substance that regulates the expression of the ABH antigen is at least one compound selected from the group consisting of 1,3-dicaffeoylquinic acid, 1,5-dicaffeoylquinic acid and amentoflavone, a derivative thereof, or a pharmaceutically acceptable salt thereof.

Hereinafter, the present invention will be described in more detail by way of examples. However, it would be obvious to those skilled in the art that these examples are for illustrative purposes only and the scope of the present invention is not construed as being limited by these examples.

### [Test Example 1] Effect of Increasing the Expression of B Antigen in HaCaT Cell Line

The effect of various kinds of compounds on the expression of ABH antigen was examined. For this purpose, HaCaT cells (provided by Prof. Dr. N E Fusenig, DKFZ Heidelberg, Germany) were cultured in 10% FBS-DMEM for 24 hours in a 35 mm dish and then cultured in 0% FBS-DMEM for 24 hours to make to a starvation state.

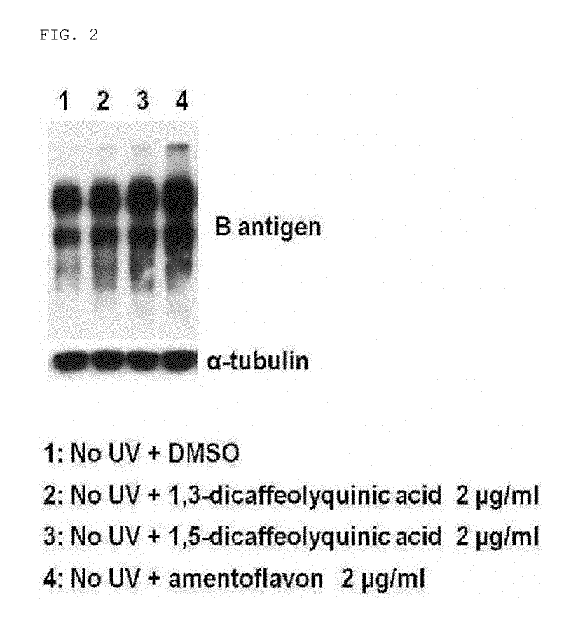

Again, while replacing the medium with 0% FBS-DMEM, various kinds of compounds as test substances were treated with HaCaT cells at a concentration of 2 μg/ml, respectively, and cultured for 48 hours. At this time, for comparison, the control group was treated with DMSO at the same concentration as the sample. Among the test substances, three substances that significantly increase the expression of ABH antigen, namely, 1,3-dicaffeoylquinic acid, 1,5-dicaffeoylquinic acid and amentoflavone were confirmed (data not shown). In case where these three compounds were treated, proteins were extracted from the cells, the cell lysate was loaded in the same amount, and then the expression of type B antigen was examined by western blot. Alpha-tubulin protein was used as a control group. The measurement results are shown in FIG. 2.

As shown in FIG. 2, it is confirmed that 1,3-dicaffeoylquinic acid, 1,5-dicaffeoylquinic acid and amentoflavone all had an effect of increasing the expression of B type antigen in HaCaT cells.

### [Test Example 2] Inhibitory Effect on 5α-Reductase Activity

In order to evaluate the inhibitory effect on the activity of 5α-reductase, the conversion rate from [¹⁴C] testosterone to [¹⁴C] dihydrotestosterone in HEK 293-5α R2 cells was measured. HEK 293 cells were transfected with p3×FLAG-CMV-5α R2, and the transfected HEK 293 cells (HEK 293-5α R2 cells) were seeded in a 24-well plate, 2.5×10⁵ cells per well (Park et al., 2003, JDS. Vol. 31, pp 191-98). The next day, the used culture medium was replaced with a new culture medium containing an enzyme substrate and an inhibitor. The substrate of the culture medium used was 0.05 μCi [¹⁴C]testosterone (Amersham Pharmacia Biotech, UK).

To evaluate the degree of 5α-reductase activity inhibition, 2 μg/ml of each of 1,3-dicaffeoylquinic acid, 1,5-dicaffeoylquinic acid and amentoflavone was added as test substances and then cultured in a 5% CO₂ incubator at 37° C. for 2 hours. At this time, for comparison, the negative control used was the group not containing any of the above test substances, while the positive control used was the group in which finasteride was added to the medium at 2 μg/ml and cultured under the same conditions. Subsequently, the culture medium was collected to extract estosterone with 800 μl of ethylacetate. The supernatant organic solvent phase was isolated and dried. The residue was dissolved in 50 μl of ethylacetate and developed on silica plastic sheet kieselgel 60 F254 using an ethylacetate-hexane (1:1) as a developing solvent.

The plastic sheet was dried out in the air and measured in regards to the abundance of isotope using a BAS system. The dry plastic sheet together with an X-ray film was put in a bath cassette. After one week, the amount of isotope of testosterone and dihydrotestosterone remaining on the film was measured. The results are presented in Table 1 below.

TABLE 1

| | Conversion | Inhibition |
|---|---|---|
| Sample | rate (%) | rate (%) |
| 1,3-dicaffeoylquinic acid | 30 | 38 |
| 1,5-dicaffeoylquinic acid | 30 | 38 |
| amentoflavone | 32 | 33 |
| Control group | 48 | — |
| Positive control group | 27 | 44 |
| (Finasteride) | | |

(1) Conversion rate: Radioactivity at DHT region/Total Radioactivity
(2) Inhibition rate: 100*(Conversion rate of control group - Conversion rate of test substance)/Conversion rate of control group

From the results in Table 1, it is confirmed that 1,3-dicaffeoylquinic acid, 1,5-dicaffeoylquinic acid and amentoflavone of the present invention could interrupt conversion of testosterone to dihydrotestosterone by effectively inhibiting the activity of 5α-reductase enzyme responsible for conversion of testosterone to dihydrotestosterone which binds to cytoplasmic receptor proteins and enters the nuclear to activate sebaceous gland cells to promote the differentiation of the sebaceous gland cells and thus cause excessive sebaceous secretions.

Therefore, 1,3-dicaffeoylquinic acid, 1,5-dicaffeoylquinic acid and amentoflavone of the present invention were effective for suppressing excessive sebaceous secretions by effectively inhibiting the activity of 5α-reductase enzyme.

### [Reference Example 1] Production of Examples 1 to 3 and Comparative Example 1

In accordance with the compositions described in Table 2 below, the lotion preparations of Examples 1 to 3 and Comparative Example 1 were prepared by a conventional method (unit: wt %).

TABLE 2

| | | Example | Example | Example | Comparative |
|---|---|---|---|---|---|
| No | Material name | 1 | 2 | 3 | Example 1 |
| 1 | Cetearyl alcohol | 1.0 | 1.0 | 1.0 | 1.0 |
| 2 | Lipophilic | 1.0 | 1.0 | 1.0 | 1.0 |
| | glyceryl stearate | | | | |
| 3 | Gltyceryl | 1.5 | 1.5 | 1.5 | 1.5 |
| | stearate SE | | | | |
| 4 | Phytosqualane | 3 | 3 | 3 | 3 |
| 5 | Hydrogenated | 2 | 2 | 2 | 2 |
| | polydecene | | | | |
| 6 | Dimethicone | 0.5 | 0.5 | 0.5 | 0.5 |
| 7 | Polysorbate 60 | 1 | 1 | 1 | 1 |
| 8 | Sorbitan | 0.4 | 0.4 | 0.4 | 0.4 |
| | sesquioleate | | | | |
| 9 | Methylparaben | 0.1 | 0.1 | 0.1 | 0.1 |
| 10 | Propylparaben | 0.05 | 0.05 | 0.05 | 0.05 |
| 11 | Purified water | To 100 | To 100 | To 100 | To 100 |
| 12 | Butylene glycol | 5 | 5 | 5 | 5 |
| 13 | Polyacrylate-13/ | 0.5 | 0.5 | 0.5 | 0.5 |
| | Polyisobutene/ | | | | |
| | Polysorbate 20 | | | | |
| 14 | 1,3- | 1 | — | — | — |
| | dicaffeoylquinic | | | | |
| | acid | | | | |
| | 1,5- | — | 1 | — | — |
| | dicaffeoylquinic | | | | |
| | acid | | | | |
| | Amentoflavone | — | — | 1 | — |

<Preparation Method of Example and Comparative Example>
1) The components 11 to 14 were uniformly mixed while heating to 70° C. to prepare an aqueous phase part.
2) The components 1 to 10 were uniformly mixed while heating to 70° C. to prepare an oil phase part.
3) The oil phase part of 2) was put into the aqueous phase part of 1) and homomixed at 7,200 rpm for 6 minutes.
4) The mixture of 3) was cooled to room temperature.

### [Test Example 3] Inhibitory Effect on Sebaceous Secretions

The procedures are performed as follows to evaluate the Examples 1-3 and the Comparative Example 1 in regards to the inhibitory effect on sebaceous secretions. 40 male or female subjects with excessive sebaceous secretions were selected and divided into 4 groups of 10 subjects each.

For each group, the lotions of Examples 1 to 3 and Comparative Example 1 were applied onto a defined region of the skin daily for 4 weeks. The determination on the effect of reducing sebaceous secretions was measured using a sebaceous secretion measurer (Sebumeter 815, Germany), and the results are shown in Table 3 below.

TABLE 3

| | Example | Example | Example | Comparative |
|---|---|---|---|---|
| | 1 | 2 | 3 | Example 1 |
| Average sebaceous | 17.2 ± 3.4 | 15.4 ± 2.5 | 18.7 ± 3.6 | 5.2 ± 2.1 |
| secretion decrement | | | | |
| (%) after 2 weeks | | | | |
| Average sebaceous | 18.5 ± 3.2 | 17.2 ± 2.8 | 20.5 ± 4.2 | 5.4 ± 2.5 |
| secretion decrement | | | | |
| (%) after 4 weeks | | | | |

From the results of Table 3, it is confirmed that Examples 1 to 3 containing the 1,3-dicaffeoylquinic acid, 1,5-dicaffeoylquinic acid and amentoflavone of the present invention could more effectively suppress excessive sebaceous secretions than the Comparative Example 1 not containing these substances.

Therefore, the skin external preparation composition according to the present invention has an excellent effect of suppressing sebaceous secretions.

### [Test Example 4] Decrease in Expression of Skin Inflammatory Factor

In order to measure the effect of suppressing the expression of PGE-2 which is a skin inflammatory factor of 1,3-dicaffeoylquinic acid, 1,5-dicaffeoylquinic acid and amentoflavone of the present invention, ELISA (Enzyme Linked ImmunoSorbent Assay) was performed (S E Dunsmore, et al., J Biol Chem, 271: 24576-24582, 1996).

5×10⁴ cells of keratinocytes isolated from human epidermal tissue were put in each well of a 24-well plate and immobilized for 24 hours. The culture medium was replaced with a medium not containing FBS and treated with aspirin to remove the activity of prostaglandin biosynthetic enzyme (prostaglandin H2 synthetase, or cyclooxygenase). Two hours after aspirin treatment, each well containing keratinocytes was washed twice with PBS to which 100 μl of PBS was added to each well. The keratinocytes were exposed to 30 mJ/cm of ultraviolet radiation under an ultraviolet B (UV B) lamp (Model: F15T8, UV B15W, Sankyo Dennki, Japan). Each well was removed of PBS and supplied with 250 ul of the keratinocyte growth media (Clonetics BioWhittacker, MD, USA).

Here, the substances 1,3-dicaffeoylquinic acid, 1,5-dicaffeoylquinic acid and amentoflavone were treated at a dose of 2 μg/ml, followed by culturing for 16 hours. By taking an appropriate amount of culture supernatant and quantifying PGE-2 biosynthesized for 16 hours, the prostaglandin inhibitory effects of 1,3-dicaffeoylquinic acid, 1,5-dicaffeoylquinic acid and amentoflavone was evaluated. The expression inhibitory effect of PGE-2 was calculated by the following mathematical formula 1, and the results are shown in Table 4 below.

Expression inhibition rate of PGE-2(%)=(A−B)/A*100 [Mathematical Formula 1]

A: Absorbance of well to which test substance was not added

B: Absorbance of the well to which test substance was added

TABLE 4

| | PGE-2 expression |
|---|---|
| Classification | inhibition rate(%) |
| Control group | — |
| 1,3-dicaffeoylquinic acid | 28.1 |
| 1,5-dicaffeoylquinic acid | 32.5 |
| Amentoflavone | 30.9 |

From the results of Table 4, it is confirmed that 1,3-dicaffeoylquinic acid, 1,5-dicaffeoylquinic acid and amentoflavone contained in the composition of the present invention effectively suppressed the expression of PGE-2, which is a skin inflammatory factor. Therefore, it can be seen that 1,3-dicaffeoylquinic acid, 1,5-dicaffeoylquinic acid and amentoflavone contained in the composition of the present invention were very effective in inhibiting the expression of skin inflammatory factor and preventing skin troubles.

### [Experimental Example 5] Effect of Inhibiting Formation of Reactive Oxygen Species

5×10⁴ cells of Keratinocytes isolated from the human epidermal tissue were put in each well of a 24-well plate and immobilized for 24 hours. After the culture medium was removed, 100 μl of phosphate buffered saline (PBS) solution was added to each well. The keratinocytes were exposed to 30 mJ/cm² of ultraviolet radiation under an ultraviolet B (UV B) lamp (Model: F15T8, UV B 15 W, Sankyo Denki Co., Ltd., Japan). Each well was removed of the PBS solution and then supplied with 200 μl1 of the keratinocyte culture medium. The well was treated with 2 μg/ml of 1,3-dicaffeoylquinic acid, 1,5-dicaffeoylquinic acid and amentoflavone as the test substances, respectively. Then, the quantity of the reactive oxygen species (ROS) increased by the UV stimulation was determined at defined time intervals. At this time, for comparison, the quantity of reactive oxygen species was also measured for those not treated with test substance (untreated) and not stimulated with ultraviolet rays and those subjected to ultraviolet stimulation without treatment of the test substance. The quantity of ROS was quantified by referring to Tan's method for measuring the fluorescence of dichlorofluorescin diacetate (DCF-DA) oxidized by ROS (Tan et al., 1998, J. Cell Biol. Vol. 141, pp 1423-1432). The calculation results of the ratio to the ROS of the control group are shown in Table 5 below.

TABLE 5

| | Elapsed time after exposure to | | |
|---|---|---|---|
| | 30 mJ/cm² of UVB | | |
| | 0 hr | 2 hrs | 3 hrs |
| untreated | 100 | 244 | 287 |
| UVB + untreated | 100 | 325 | 381 |
| UVB + 1,3- | 100 | 277 | 319 |
| dicaffeoylquinic | | | |
| acid | | | |
| UVB + 1,5- | 100 | 280 | 315 |
| dicaffeoylquinic | | | |
| acid | | | |
| UVB + amentoflavone | 100 | 288 | 320 |

From the results of Table 5, it is confirmed that 1,3-dicaffeoylquinic acid, 1,5-dicaffeoylquinic acid and amentoflavone of the present invention effectively suppressed the generation of ROS known to cause damages on the skin cells under UV radiation and thus these substances were excellent in antioxidative efficacy.

Therefore, it can be seen that 1,3-dicaffeoylquinic acid, 1,5-dicaffeoylquinic acid and amentoflavone contained in the composition of the present invention not only suppressed the generation of the reactive oxygen species and inhibited skin inflammation, thereby defending against skin irritation production, but also prevented the skin cells from being damaged and prevented skin aging, thereby preventing skin pores from getting widen.

### [Experimental Example 6] Promotion of Collagen Biosynthesis

The collagen biosynthesis promoting effect of 1,3-dicaffeoylquinic acid, 1,5-dicaffeoylquinic acid and amentoflavone used in the present invention was measured in comparison with TGF-β.

First, 1×10⁵ cells of fibroblasts were seeded in each well of a 24-well plate and cultured until they grew to about 90%. This was cultured in a serum-free DMEM medium for 24 hours and then treated with 2 μg/ml of 1,3-dicaffeoylquinic acid, 1,5-dicaffeoylquinic acid, amentoflavone and TGF-β, respectively, and cultured for 24 hours in a CO₂ incubator. These supernatant fluids were removed and the procollagen type(I) ELISA kit was used to observe whether procollagen was increased or decreased. The results are shown in Table 6, and the collagen synthesis ability was compared with the untreated group as 100.

TABLE 6

| | Collagen synthesis |
|---|---|
| Classification | ability (%) |
| Untreated group | 100 |
| TGF-beta | 183.5 ± 13.1 |
| 1,3-dicaffeoylquinic | 142.1 ± 13.1 |
| acid | |
| 1,5-dicaffeoylquinic | 144.2 ± 11.0 |
| acid | |
| Amentoflavone | 147.7 ± 15.8 |

From the results shown in Table 6, it is confirmed that the 1,3-dicaffeoylquinic acid, 1,5-dicaffeoylquinic acid and amentoflavone of the present invention exhibited high collagen synthesis ability like TGF-beta as a positive control.

Therefore, it can be seen that the 1,3-dicaffeoylquinic acid, 1,5-dicaffeoylquinic acid and amentoflavone of the present invention could reduce the widened skin pores by increasing the amount of collagen produced around the skin pores.

### [Experimental Example 7] Evaluation of the Effect of Reducing Skin Pores

The skin pore reducing effect of 1,3-dicaffeoylquinic acid, 1,5-dicaffeoylquinic acid and amentoflavone were measured in comparison with tocopherol and EGCG. Sixty rhino mice were divided into six groups of 10 animals, and 0.5 ml of 1% solution of 1,3-dicaffeoylquinic acid, 1,5-dicaffeoylquinic acid and amentoflavone, tocopherol or EGCG (using 1,3-butylene glycol:ethanol=7:3 as a solvent) was applied to each group of rhino mice. At this time, for comparison, only 0.5 ml of solvent was applied to one group. The substances were treated for 1 week and the back part was biopsied 24 hours after the last treatment. The epidermis was separated, immersed in 0.5% acetic acid, fixed in 10% formalin and cut vertically at 6 mm. After staining with hematoxylin and eosin, the skin pore size was measured using a mechanical eyepiece micrometer. The results are shown in Table 7 below.

TABLE 7

| | Skin Pore |
|---|---|
| Substance | size (mm) |
| Control group | 65 |
| Tocopherol | 64 |
| EGCG | 60 |
| 1,3-dicaffeoylquinic acid | 53 |
| 1,5-dicaffeoylquinic acid | 54 |
| Amentoflavone | 50 |

As shown in Table 7, it is confirmed that the 1,3-dicaffeoylquinic acid, 1,5-dicaffeoylquinic acid and amentoflavone of the present invention were excellent in the effect of decreasing the skin pore size compared to tocopherol and EGCG.

### [Reference Example 2] Preparation of Examples 4 to 6 and Comparative Example 2

The softening lotion (skin lotion) preparations of Examples 4 to 6 and Comparative Example 2 were prepared by a conventional method (unit: % by weight) according to the composition shown in Table 8 below.

TABLE 8

| | Example | Example | Example | Comparative |
|---|---|---|---|---|
| Mixed component | 1 | 2 | 3 | Example 1 |
| Purified water | Balance | Balance | Balance | Balance |
| 1,3-dicaffeoylquinic | 0.1 | — | — | — |
| acid | | | | |
| 1,5-dicaffeoylquinic | — | 0.1 | — | — |
| acid | | | | |
| Amentoflavone | — | — | 0.1 | — |
| Butylene glycol | 2.0 | 2.0 | 2.0 | 2.0 |
| Propylene glycol | 2.0 | 2.0 | 2.0 | 2.0 |
| Carboxy vinyl polymer | 0.1 | 0.1 | 0.1 | 0.1 |
| PEG-12 nonylphenyl | 0.2 | 0.2 | 0.2 | 0.2 |
| ether | | | | |
| Polysorbate 80 | 0.4 | 0.4 | 0.4 | 0.4 |
| Ethanol | 10.0 | 10.0 | 10.0 | 10.0 |
| Triethanol amine | 0.1 | 0.1 | 0.1 | 0.1 |
| Antiseptic, pigment, | q.s. | q.s. | q.s. | q.s. |
| fragrance | | | | |

### [Experimental Example 8] Sensory Evaluation on Skin Pore Reduction

For the subjects to be tested, 60 females from 20 to 50 years old with oily skin were divided into 4 groups of 15 females randomly. After lapse of a predetermined time after facial cleansing for each group, the products of Examples 4 to 6 or Comparative Example 2 were applied respectively, and the application was carried out twice a day in the morning and night, then after 4 weeks, the skin pore size was measured with the naked eye. The results are shown in Table 9 below (evaluation grade: 0. not contracted at all; 5. extremely contracted).

TABLE 9

| Classification | Evaluation grade |
|---|---|
| Example 1 | 3.3 |
| Example 2 | 3.2 |
| Example 3 | 3.7 |
| Comparative Example 1 | 0.8 |

From the results in Table 9 above, it is confirmed that there were significantly more users who answered that the composition containing 1,3-dicaffeoylquinic acid, 1,5-dicaffeoylquinic acid and amentoflavone of the present invention exhibited superior skin pore contracting effect compared with the composition not containing any of these compounds.

Therefore, it was found that the skin pore reduction effect of the external skin preparation composition containing 1,3-dicaffeoylquinic acid, 1,5-dicaffeoylquinic acid and amentoflavone of the present invention was excellent.

Examples of formulations of the composition according to the present invention will be described below, but the pharmaceutical composition and the cosmetic composition can be applied in various dosage forms, which are for illustrative only and the scope of the present invention is not limited thereto.

### [Preparation Example 1] Skin Lotion

Skin lotion was prepared by a conventional method using the composition described in Table 10 below.

TABLE 10

| | Content (wt %) |
|---|---|
| At least one compound selected | 2.0 |
| from the group consisting of | |
| 1,3-dicaffeoylquinic acid, 1,5- | |
| dicaffeoylquinic acid and | |
| amentoflavone | |
| Glycerin | 3.0 |
| Butylene glycol | 2.0 |
| Propylene glycol | 2.0 |
| Carbloxy vinyl polymer | 0.1 |
| PEG 12 nonylphenyl ether | 0.2 |
| Polysorbate 80 | 0.4 |
| Ethanol | 10.0 |
| Triethanol amine | 0.1 |
| Antiseptic, pigment, fragrance | q.s. |
| Purified water | balance |

### [Preparation Example 2] Nutrition Cream

Nutrition cream was prepared by a conventional method using the composition described in Table 11 below.

TABLE 11

| | Content (wt %) |
|---|---|
| At least one compound selected | 2.0 |
| from the group consisting of | |
| 1,3-dicaffeoylquinic acid, | |
| 1,5-dicaffeoylquinic acid and | |
| amentoflavone | |
| Polysorbate 60 | 1.5 |
| Sorbitan sesquioleate | 0.5 |
| PEG 60 hydrogenated castor oil | 2.0 |
| Liquid paraffin | 10.0 |
| Squalane | 5.0 |
| Caprylic/capric triglyceride | 5.0 |
| Glycerin | 5.0 |
| Butylene glycol | 3.0 |
| Propylene glycol | 3.0 |
| Triethanolamine | 0.2 |
| Antiseptic, pigment, fragrance | q.s. |
| Purified water | balance |

### [Preparation Example 3] Massage Cream

Massage cream was prepared by a conventional method using the composition described in Table 12 below.

TABLE 12

| | Content(wt %) |
|---|---|
| At least one compound selected | 1.0 |
| from the group consisting of | |
| 1,3-dicaffeoylquinic acid, | |
| 1,5-dicaffeoylquinic acid and | |
| amentoflavone | |
| Wax | 10.0 |
| Polysorbate 60 | 1.5 |
| PEG 60 hydrogenated castor oil | 2.0 |
| Sorbitan sesquioleate | 0.8 |
| Liquid paraffin | 40.0 |
| Squalane | 5.0 |
| Caprylic/capric triglyceride | 4.0 |
| Glycerin | 5.0 |
| Butylene glycol | 3.0 |
| Propylene glycol | 3.0 |
| Triethanolamine | 0.2 |
| Antiseptic, pigment, fragrance | q.s. |
| Purified water | Balance |

### [Preparation Example 4] Pack

Pack was prepared by a conventional method using the composition described in Table 13 below.

TABLE 13

| | Content (wt %) |
|---|---|
| At least one compound | 1.0 |
| selected from the group | |
| consisting of 1,3- | |
| dicaffeoylquinic acid, 1,5- | |
| dicaffeoylquinic acid and | |
| amentoflavone | |
| Polyvinylalcohol | 13.0 |
| Sodium carboxymethylcellulose | 0.2 |
| Glycerin | 5.0 |
| Allantoin | 0.1 |
| Ethanol | 6.0 |
| PEG 12 nonylphenyl ether | 0.3 |
| Polysorbate 60 | 0.3 |
| Antiseptic, pigment, fragrance | q.s. |
| Purified water | Balance |

### [Preparation Example 5] Gel

Gel was prepared by a conventional method using the composition described in Table 14 below.

TABLE 14

| | Content(wt %) |
|---|---|
| At least one compound selected | 0.5 |
| from the group consisting of | |
| 1,3-dicaffeoylquinic acid, | |
| 1,5-dicaffeoylquinic acid and | |
| amentoflavone | |
| Ethylenediamine sodium acetate | 0.05 |
| Glycerin | 5.0 |
| Caroboxy vinyl polymer | 0.3 |
| Ethanol | 5.0 |
| PEG 60 hydrogenated castor oil | 0.5 |
| Triethanolamine | 0.3 |
| Antiseptic, pigment, fragrance | q.s. |
| Purified water | Balance |

### [Preparation Example 6] Ointment

Ointment was prepared by a conventional method using the composition described in Table 15 below.

TABLE 15

| | Content(wt %) |
|---|---|
| At least one compound selected | 0.1 |
| from the group consisting of | |
| 1,3-dicaffeoylquinic acid, 1,5- | |
| dicaffeoylquinic acid and | |
| amentoflavone | |
| Glycerin | 8.0 |
| Butylene glycol | 4.0 |
| Liquid paraffin | 15.0 |
| Beta glucan | 7.0 |
| Carbomer | 0.1 |
| Caprylic/capric triglyceride | 3.0 |
| Squalane | 1.0 |
| Cetearyl glucoside | 1.5 |
| Sorbitan stearate | 0.4 |
| Cetearyl alcohol | 1.0 |
| Wax | 4.0 |
| Antiseptic, pigment, fragrance | q.s. |
| Purified water | Balance |

### [Preparation Example 7] Soft Capsule

A soft capsule was prepared by mixing 0.0025 g of at least one compound selected from the group consisting of 1,3-dicaffeoylquinic acid, 1,5-dicaffeoylquinic acid and amentoflavone, 0.0025 g of vitamin C, 2 mg of palm oil, 8 mg of palm kernel oil, 4 mg of yellow wax and 6 mg of lecithin, and filling each 400 mg per capsule by the usual method.

### [Preparation Example 8] Tablet

0.0025 g of at least one compound selected from the group consisting of 1,3-dicaffeoylquinic acid, 1,5-dicaffeoylquinic acid and amentoflavone, 0.0025 g of vitamin C, 100 mg of glucose, 96 mg of starch to which 4 mg of magnesium stearate were mixed and 40 mg of 30% ethanol was added to form granules, The granules were dried at 60° C. and tableted into tablet using a tableting machine.

### [Preparation Example 9] Granules

150 mg of at least one compound selected from the group consisting of 1,3-dicaffeoylquinic acid, 1,5-dicaffeoylquinic acid and amentoflavone, 150 mg vitamin C, 100 mg of glucose and 600 mg of starch were mixed to which 100 mg of 30% ethanol was added to form granules. The granules were dried at 60° C. to form granules, which were then filled in a capsule. The final weight of the contents was 1 g.

### [Preparation Example 10] Drinks

0.0025 mg of at least one compound selected from the group consisting of 1,3-dicaffeoylquinic acid, 1,5-dicaffeoylquinic acid and amentoflavone, 0.0025 g of vitamin C, 10 g of glucose, 2 g of citric acid and 187.8 g of purified water were mixed and filled in a bottle. The final dose of the contents was 200 ml.

Although specific portions of the contents of the present invention have been described in detail above, it would be obvious to those skilled in the art that such specific techniques are merely preferred embodiments and the scope of the present invention is not limited thereto. It is therefore to be understood that the substantial scope of the invention is defined by the claims and their equivalents.

1. A method of reducing a skin pore size or inhibiting a skin pore enlargement of the skin of a subject in need thereof, comprising applying an effective amount of a composition containing a substance increases the expression of an ABH antigen,
wherein the substance that increases the expression of the ABH antigen is at least one compound selected from the group consisting of 1,3-dicaffeoylquinic acid, 1,5-dicaffeoylquinic acid and amentoflavone, a derivative thereof, or a pharmaceutically or cosmetically acceptable salt thereof.

2. The method of claim 1, wherein the composition contains the substance that increases the expression of ABH antigen in an amount of 0.001% by weight to 20% by weight based on the total weight of the composition.

3. The method of claim 1, wherein the composition is an external preparation for skin.

4. The method of claim 1, wherein the composition is a cosmetic composition or a pharmaceutical composition.

5. The method of claim 1, wherein the composition is in a formulation selected from the group consisting of suspension, ointment, lotion, and gel formulations.

6. A method for screening a substance capable of reducing a skin pore size or inhibiting a skin pore enlargement, comprising steps of:
measuring an expression level of a ABO blood group (ABH) antigen expressed in test skin cells cultured in a culture medium in the absence of a candidate substance;
culturing the test skin cells in the culture medium in the presence of a candidate substance under the same condition;
measuring the expression level of the ABH antigen from the test skin cells of step 2); and
comparing the expression levels of the steps 1) and 3) to determine whether the candidate substance increases the expression of the ABH antigen,
wherein an increased expression level of the step 3) than the step 1) indicates that the tested candidate substance is capable of reducing a skin pore size or inhibiting a skin pore enlargement.

7. The screening method of claim 6, wherein the substance capable of reducing skin pore size or inhibiting skin pore enlargement is at least one compound selected from the group consisting of 1,3-dicaffeoylquinic acid, 1,5-dicaffeoylquinic acid and amentoflavone, or a pharmaceutically acceptable salt thereof.
